(19) United States
(12) Patent Application Publication
(10) Pub. No.: US 2025/0107768 A1
(43) Pub. Date: Apr. 3, 2025

(54) IMAGING SUPPORT DEVICE, RECORDING MEDIUM, IMAGING SUPPORT METHOD, AND IMAGING SUPPORT SYSTEM

(71) Applicant: Konica Minolta, Inc., Tokyo (JP)

(72) Inventor: Ryohei ITO, Tokyo (JP)

(21) Appl. No.: 18/890,001

(22) Filed: Sep. 19, 2024

(30) Foreign Application Priority Data
Sep. 29, 2023 (JP) 2023-169682

(51) Int. Cl. A61B 6/00

(52) U.S. Cl. CPC A61B 6/5229

(57) ABSTRACT

An imaging support device includes a hardware processor. The hardware processor, acquires first imaging support information which is first information for changing a position of a subject or an imaging device configured to image a medical image of the subject, changes the first imaging support information to second imaging support information which is second information for changing a position of the subject or the imaging device, and outputs the second imaging support information.

### CROSS-REFERENCE TO RELATED APPLICATIONS

The entire disclosure of Japanese Patent Application No. 2023-169682, filed on Sep. 29, 2023, including description, claims, drawings and abstract is incorporated herein by reference.

### BACKGROUND OF THE INVENTION

### Technical Field

The present invention relates to an imaging support device, a recording medium, an imaging support method, and an imaging support system.

### Description of Related Art

In a case where there is a shift in positioning of a patient in radiography, correction to correct positioning is performed. When the positioning is corrected, technical terms such as internal rotation, external rotation, adduction, abduction, and the like indicating the correction direction of the positioning are generally used. However, even an expert such as a radiologist may have difficulty in instantaneously understanding technical terms such as internal rotation, external rotation, adduction, and abduction when viewing or hearing the terms.

Conventionally, a technology for converting a term into another expression has been disclosed. Japanese Unexamined Patent Publication No. 2019-36333 discloses the following text processing apparatus. The text processing apparatus converts the word extracted from the sentence by the dictionary accessed according to the environment information, and writes the converted word together with the word of the sentence. Japanese Unexamined Patent Publication No. 2005-338970 describes the following text processing program. The text processing program selects, from among the words obtained by dividing the text data, a word included in the technical term list as a word to be replaced. Next, the text processing program replaces each of the selected replacement object words with a word included in the general-purpose word list.

However, in the conventional technology, a word or the like after conversion may not be a phrase or the like that each user is accustomed to. In addition, conventionally, there is a problem in that a technique capable of converting a technical term into an expression preferred by a user does not exist in the field of radiography.

### SUMMARY OF THE INVENTION

Therefore, it is an object of the present invention to provide an imaging support device, a recording medium including a program, an imaging support method, and an imaging support system that enable a user to select information for supporting a correction direction of positioning.

The imaging support device according to the present invention includes a hardware processor, wherein the hardware processor, acquires first imaging support information which is first information for changing a position of a subject or an imaging device configured to image a medical image of the subject, changes the first imaging support information to second imaging support information which is second information for changing a position of the subject or the imaging device, and outputs the second imaging support information.

A non-transitory computer-readable recording medium according to the present invention stores a program that causes a computer to perform: acquiring first imaging support information which is first information for changing a position of a subject or an imaging device configured to image a medical image of the subject, changing the first imaging support information to second imaging support information which is second information for changing a position of the subject or the imaging device, and outputting the second imaging support information.

An imaging support method according to the present invention includes, acquiring first imaging support information which is first information for changing a position of a subject or an imaging device configured to image a medical image of the subject, changing the first imaging support information to second imaging support information which is second information for changing a position of the subject or the imaging device, and outputting the second imaging support information.

An imaging support system according to the present invention includes, an imaging device that images a subject or a medical image of the subject, and the imaging support device according to the aspect 1.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a diagram illustrating a schematic configuration of an imaging support system according to the present embodiment;

FIG. 2 is a block diagram of an imaging control device according to the present embodiment;

FIG. 3 is a diagram illustrating an example of a configuration of a candidate table stored in a storage section according to the present embodiment;

FIG. 4A is a diagram for explaining actions of external rotation and internal rotation;

FIG. 4B is a diagram for explaining actions of abduction and adduction;

FIG. 5A is a diagram illustrating an example of a configuration of an imaging support information setting screen according to the present embodiment;

FIG. 5B is a diagram illustrating an example of a state in which a selection section is expanded on an imaging support information setting screen according to the present embodiment;

FIG. 6 is a diagram illustrating an example of a configuration of a list stored in a storage section according to the present embodiment;

FIG. 7 is a diagram illustrating an example of an analysis result of a positioning shift in a radiation image of a “knee (right knee) joint side surface” according to the related art;

FIG. 8A is a diagram illustrating an example of input data used for training a machine learning model according to the present embodiment;

FIG. 8B is a diagram illustrating an example of ground truth data used for training a machine learning model according to the present embodiment;

FIG. 9 is a diagram illustrating an example of other ground truth data for training the machine learning model according to the present embodiment;

FIG. 10 is a flowchart illustrating an example of operations of the imaging control device at the time of capturing a radiation image of a subject according to the present embodiment;

FIG. 11 is a diagram illustrating an example of a configuration of an imaging screen displayed on a display part according to the present embodiment;

FIG. 12 is a flowchart illustrating an example of the operations of the imaging control device at the time of re-imaging determination processing according to the present embodiment;

FIG. 13A is a diagram illustrating an example of three-dimensional information of a radiation image inferred from the machine learning model according to the present embodiment;

FIG. 13B is a diagram illustrating an example of three-dimensional information of a radiation image inferred from the machine learning model according to the present embodiment;

FIG. 14 is a flowchart illustrating an example of a first determination process according to the present embodiment;

FIG. 15 is a flowchart illustrating an example of a second determination process according to the present embodiment;

FIG. 16 is a diagram illustrating an example of second imaging support information and the three-dimensional structure information displayed on an imaging screen according to the present embodiment;

FIG. 17 is a diagram showing an example of first imaging support information and the three-dimensional structure information displayed on the imaging screen according to the present embodiment;

FIG. 18 is a diagram illustrating an example of the second imaging support information and the three-dimensional structure information in a case where a positioning shift in one direction displayed on the imaging screen according to the present embodiment is corrected;

FIG. 19 is a diagram illustrating an example of the second imaging support information and the three-dimensional structure information for changing the position of the imaging device displayed on the imaging screen according to the present embodiment; and

FIG. 20 is a diagram illustrating a schematic configuration of an imaging support system according to another Embodiment 1.

### DETAILED DESCRIPTION

Hereinafter, one or more embodiments of the present invention will be described with reference to the drawings. However, the scope of the invention is not limited to the disclosed embodiments.

In the following, preferred embodiments of the present disclosure will be described in detail with reference to the accompanying drawings.

### Configuration Example of Imaging Support System 10A

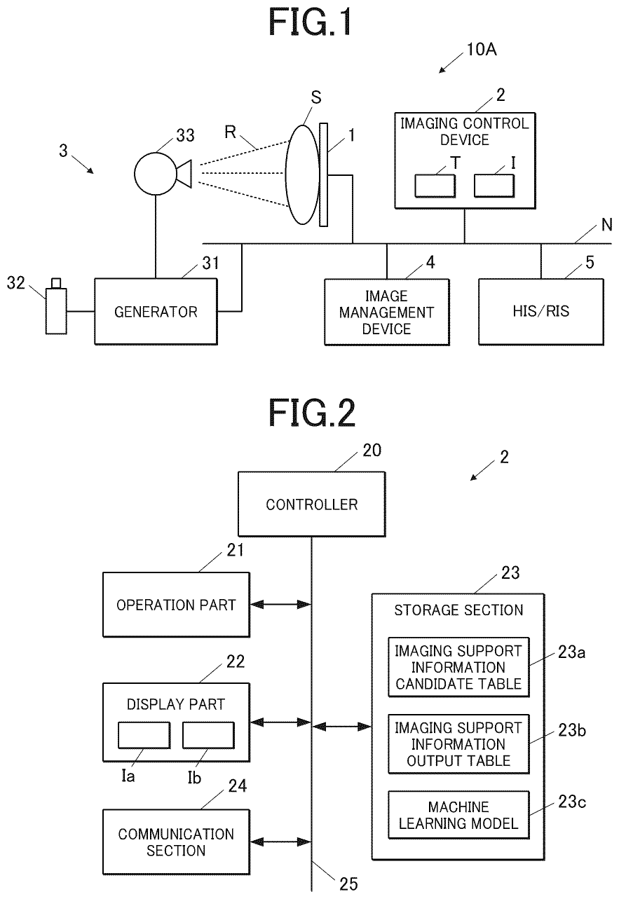

FIG. 1 is a diagram illustrating a schematic configuration of an imaging support system 10A according to the present embodiment. The imaging support system 10A includes a radiographic imaging device 1, an imaging control device 2, a radiation generation device 3, an image management device 4, and a HIS/RIS 5. Hereinafter, the radiographic imaging device may be referred to as the imaging device 1, and the radiation generation device may be referred to as the generation device 3. HIS is an abbreviation for Hospital Information System and is a hospital information system. RIS is an abbreviation for Radiology Information System and is a radiology department information system. The radiation image is an example of a medical image. The imaging control device 2 is an example of an imaging support device.

The imaging device 1, the imaging control device 2, the generation device 3, the image management device 4, and the HIS/RIS 5 are communicably connected to each other via a network N. Examples of the network N include a LAN, a WAN, or the Internet. LAN is an abbreviation for Local Area Network. WAN is an abbreviation for Wide Area Network. A communication system of the network N may be wired communication or wireless communication. The wireless communication includes, for example. Wi-Fi (registered trademark).

The generation device 3 includes a generator 31, a switch 32, and a radiation source 33. The generator 31 applies, to the radiation source 33 including, for example, a tube, a voltage according to imaging conditions set in advance, in response to operation of the switch 32. The generator 31 may include an operation part that receives input of irradiation conditions and the like.

When the generator 31 applies a voltage, the radiation source 33 generates radiation R having a dose according to the applied voltage. The radiation R is, for example, X-rays.

The generation device 3 generates the radiation R in a manner corresponding to the type of radiation image, for example, a still image or a dynamic image. To be specific, in generating a still image, the generation device 3 emits the radiation R only once in response to the switch 32 being pressed once. In generating a dynamic image, for example, in response to the switch 32 being pressed once, the generation device 3 repeatedly emits pulsed radiation R multiple times per predetermined period of time.

The imaging device 1 generates digital image data in which an imaging site of the subject S is captured. For the imaging device 1, for example, a portable FPD is used. FPD is an abbreviation for Flat Panel Detector. The imaging device 1 may be integrated with the generation device 3.

Although not illustrated, the imaging device 1 includes, for example, an imaging element, a sensor substrate, a scanner, a reader, a controller, and a communication section. Upon receiving the radiation R, the imaging element generates charges corresponding to the dose. The switch elements are two-dimensionally arranged in the sensor substrate, and the sensor substrate accumulates and discharges electric charge. The scanner switches on and off of each switch element. The reader reads, as a signal value, an amount of electric charge emitted from each pixel. The controller generates image data of the radiation image from the plurality of signal values read by the reader. The image data includes still image data or dynamic image data. The communication section transmits the generated image data, various signals, and the like to other devices such as the imaging control device 2, and receives various kinds of information and various signals from other devices.

The imaging control device 2 sets imaging conditions for the imaging device 1, the generation device 3, and the like, and controls a reading operation of the radiation image imaged by the imaging device 1. The imaging control device 2 is also referred to as a console, and is constituted by, for example, a personal computer. The imaging control device 2 determines whether or not re-imaging is necessary according to positioning shift of the radiation image obtained by imaging. Here, the positioning means, for example, how to position a patient posture during imaging. When determining that re-imaging is necessary, the imaging control device 2 causes a display part 22 (described later) to display imaging support information I and three-dimensional structure information T on its screen. The imaging support information I assists positioning correction by presenting a direction in which the positioning is to be corrected (direction of positioning correction) or the like in the form of words or sentences. Note that the imaging support information I includes first imaging support information Ia and second imaging support information Ib, as described later. The three-dimensional structure information T indicates a three-dimensional positional relationship of a shift region in which the shift of the radiation image occurs, thereby presenting basis information for changing the positioning.

The imaging conditions include, for example, patient conditions related to a subject S, irradiation conditions related to the emission of the radiation R, and image reading conditions related to the image reading of the imaging device 1. The patient conditions include, for example, an imaging site, an imaging direction, and a physique. The irradiation conditions are, for example, tube voltage (kV), tube current (mA), irradiation time (ms), current-time product (mAs value), and the like. The image reading conditions include, for example, a pixel size, an image size, and a frame rate. The imaging control device 2 may automatically set the imaging conditions on the basis of the order information acquired from the HIS/RIS 5 or the like. The imaging control device 2 may set the imaging conditions in response to manual operations by a user such as a radiologist on an operation part 21 described later.

The image management device 4 manages the image data generated by the imaging device 1. The image management device 4 is a picture archiving and communication system, a diagnostic imaging workstation, or the like. The picture archiving and communication system may be referred to as PACS. PACS is an abbreviation for Picture Archiving and Communication System.

The HIS/RIS 5 receives, for example, the order information on the radiographing of the patient from a doctor or the like, and transmits the received order information to the imaging control device 2. The order information includes, for example, various kinds of information such as an ID, an imaging site, an imaging direction, and a physique of the patient.

### Example of Configuration of Block Diagram of Imaging Control Device 2

FIG. 2 is a block diagram of the imaging control device 2. The imaging control device 2 includes a controller 20 (hardware processor), an operation part 21, a display part 22, a storage section 23 (storage), and a communication section 24. The controller 20, the operation part 21, the display part 22, the storage section 23, and the communication section 24 are communicably connected via, for example, a bus 25.

The controller 20 includes, for example, a processor such as a CPU and a semiconductor memory. CPU is an abbreviation for Central Processing Unit. The processor implements various kinds of processing including imaging control and re-imaging determination by executing various kinds of programs stored in a semiconductor memory such as a RAM (which may be the storage section 23). The controller 20 may include an electronic circuit such as an ASIC or an FPGA. ASIC is an abbreviation for Application Specific Integrated Circuit. FPGA is an abbreviation for Field Programmable Gate Array.

The controller 20 functions as an acquisition section (acquisition step), a change section (change step), and an output section (output step). The controller 20 functions as a setting section and a determination section. Each function of the acquisition section, the change section, the output section, the setting section, and the determination section is performed by the processor of the controller 20 executing a program stored in the storage section 23 or the like.

The acquisition section acquires first imaging support information Ia which is first information for changing the position of the subject S or the imaging device 1. The first imaging support information Ia corresponds to first re-imaging support information for supporting re-imaging of the radiation image. The first imaging support information Ia includes, for example, words or sentences including technical terms. Specifically, the word or sentence included in the first imaging support information Ia is at least one of adduction, abduction, internal rotation, and external rotation. The first imaging support information Ia may be set in advance by initial setting, or may be set by an input operation on imaging support information setting screen 70 which will be described later, for example. The first imaging support information Ia includes information for changing the position of at least one of the radiation source 33 and the imaging device 1 in addition to information for correcting the positioning of the patient.

The setting section sets a word or a sentence after replacement from among a plurality of replacement candidates corresponding to a word or a sentence before replacement included in the first imaging support information. The word or sentence after replacement, which is the second imaging support information Ib described later, may be set, for example, by an input operation on a setting screen described later. The change section changes the first imaging support information Ia to the second imaging support information Ib which is second information for changing the position of the subject S or the imaging device 1. The changing section changes the first imaging support information Ia to the second imaging support information Ib on the basis of the replaced word or sentence set by the setting section. The second imaging support information Ib corresponds to second re-imaging support information for supporting re-imaging of the radiation image, and is information obtained by changing the expression of the first imaging support information Ia. The second imaging support information Ib does not include technical terms, and is constituted by words or sentences that are easy for the user to understand. The second imaging support information Ib also includes information for changing the position of at least one of the radiation source 33 and the imaging device 1 in addition to information for correcting the positioning of the patient.

The output section outputs the first imaging support information Ia or the second imaging support information Ib. Specifically, when the first imaging support information Ia is set by the user, the output section outputs the first imaging support information Ia to the display part 22. When the second imaging support information Ib is set by the user, the output section outputs the second imaging support information Ib to the display part 22.

The determination section determines a shift of a predetermined region in the radiation image obtained by imaging of the imaging device 1. The determination section may include a first determination section that determines a first shift of the predetermined region in the radiation image, and a second determination section that determines a second shift of the predetermined region in the radiation image. The first shift and the second shift correspond to respective correction directions of the positioning. The deriving section derives the first imaging support information Ia or the second imaging support information Ib on the basis of the determination of the shift by the determination section.

The operation part 21 receives a command according to various input operations by a user, converts the received command into an operation signal, and outputs the operation signal to the controller 20. The operation part 21 includes, for example, at least one of a mouse, a keyboard, a switch, and a button. The operation part 21 may be, for example, a touch screen integrally combined with a display. The operation part 21 may be, for example, a user interface such as a microphone that receives a voice input. Specifically, the operation part 21 receives a change instruction or the like for changing the assist information of the correction direction of the positioning from the first imaging support information to the second imaging support information.

The display part 22 displays a radiation image based on image data received from the imaging device 1, a GUI for receiving various input operations from the user, and the like. GUI is an abbreviation for Graphical User Interface. The display part 22 is, for example, a display such as a liquid crystal display or an organic EL display. EL is an abbreviation for Electro Luminescence. Specifically, the display part 22 displays the radiation image obtained by imaging of the imaging device 1, and displays the imaging support information I and the three-dimensional structure information T according to the result of the re-imaging determination processing.

The storage section 23 stores, for example, a system program, an application program, and various types of data. The storage section 23 includes, for example, any storage module such as an HDD, an SSD, a ROM, and a RAM. HDD is an abbreviation for Hard Disk Drive. SSD is an abbreviation for Solid State Drive. ROM is an abbreviation for Read Only Memory. To be specific, the storage section 23 stores an imaging support information candidate table 23a, an imaging support information output table 23b, and a machine learning model (learned model) 23c. The machine learning model 23c and the like may be stored in an externally provided storage device or the like. Note that details of the imaging support information candidate table 23a, the imaging support information output table 23b, and the machine learning model (learned model) 23c will be described later.

The communication section 24 includes, for example, a communication module including an NIC, a receiver, and a transmitter. NIC is an abbreviation for Network Interface Card. The communication section 24 communicates various types of data such as image data among the imaging device 1, the image management device 4, and the like through the network N.

Note that the imaging control device 2 may be configured not to include the operation part 21 and the display part 22. In that case, the imaging control device 2 may receive control signals from an operation part provided in an external device connected via the communication section 24. The imaging control device 2 may output an image signal to a display part provided in the external device to display a radiation image or the like. The external device may be the image management device 4 or the like, or may be another device.

### Example of Configuration of Imaging Support Information Candidate Table 23a

Next, an example of the configuration of the imaging support information candidate table 23a stored in the storage section 23 will be described. Hereinafter, the imaging support information candidate table 23a may be referred to as a candidate table 23a.

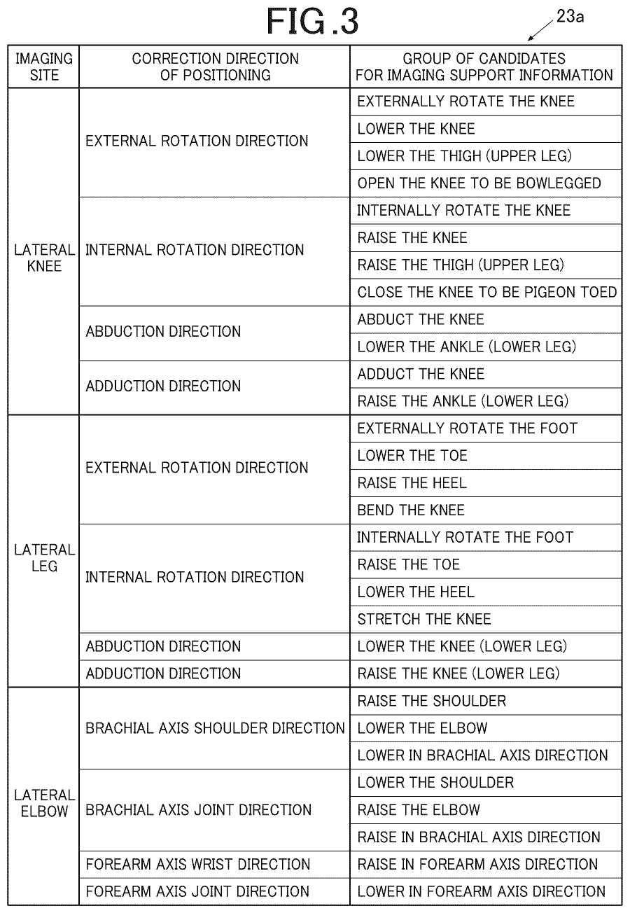

FIG. 3 illustrates an example of the configuration of the candidate table 23a. In the candidate table 23a, the imaging site and the imaging direction, the correction direction in the case of correcting the shift of the positioning, and a candidate group of imaging support information I for presenting a specific correction direction of the positioning are stored in association with each other. Examples of the imaging site and the imaging direction include “lateral knee joint”, “lateral leg”, and “lateral elbow”.

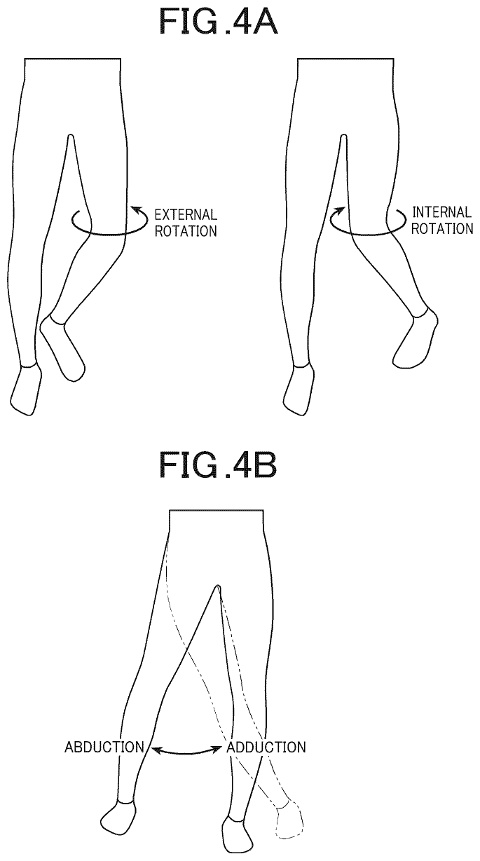

Examples of the type of direction of correction in the case of correcting a positioning shift include “external rotation”, “internal rotation”, “abduction”, and “adduction”. FIG. 4A is a diagram for explaining actions of external rotation and internal rotation. FIG. 4B is a diagram for explaining actions of abduction and adduction. As illustrated on the left side of FIG. 4A, external rotation is a movement of rotating the femur outward with respect to the long axis of the bone. Internal rotation is, as illustrated on the right side of FIG. 4A, a movement of rotating the femur inward with respect to the long axis of the bone. Abduction is the movement of the femur away from the central axis of the body, as shown in FIG. 4B. Adduction is the movement of the femur toward the central axis of the body, as shown in FIG. 4B.

The candidate group of the imaging support information I includes first imaging support information Ia including a word or sentence before replacement and second imaging support information Ib including a word or sentence after replacement corresponding to the first imaging support information Ia. Specifically, when the imaging site and the imaging direction are “lateral knee joint” and the correction direction of the positioning is “external rotation”, an arbitrary sentence can be selected from the candidate group including four pieces of imaging support information I. Candidates for the first imaging support information Ia include, for example. “externally rotate the knee.” Examples of the candidate group for the second imaging support information Ib include “lower the knee,” “lower the thigh (upper leg)”, and “open the knee to be bowlegged.”

When the imaging site and the imaging direction are “lateral knee joint” and the correction direction of the positioning is “internal rotation”, an arbitrary sentence can be selected from the candidate group including four pieces of imaging support information I. Candidates for the first imaging support information Ia include, for example. “internally rotate the knee”. Examples of the group of candidates for the second imaging support information Ib include “raise the knee”, “raise the thigh (upper leg)”, and “close the knee to be pigeon toed”.

A plurality of candidate tables 23a may be provided. For example, a user dictionary in which assist information on the direction of correction of positioning frequently used by the user is registered may be stored as a table in the storage section 23. For example, a table may be created for each region, such as a Kansai-zone dictionary in which assist information used in the Kansai-zone is registered and a Kanto-zone dictionary in which assist information used in the Kanto-zone is registered. In addition, a table may be created for each hospital or facility, such as a university hospital dictionary in which assist information used in each university hospital is registered. The plurality of candidate tables 23a constitute an example of a first replacement candidate and a second replacement candidate. For example, a sentence or the like after replacement corresponding to a sentence or the like before replacement is set from among the plurality of candidate tables 23a, and the first imaging support information can be changed to the second imaging support information based on the set sentence or the like.

### Example of Configuration of Imaging Support Information Setting Screen 70

Next, the imaging support information setting screen 70 for setting arbitrary first imaging support information Ia and second imaging support information Ib from a candidate group in a plurality of imaging support information I will be described.

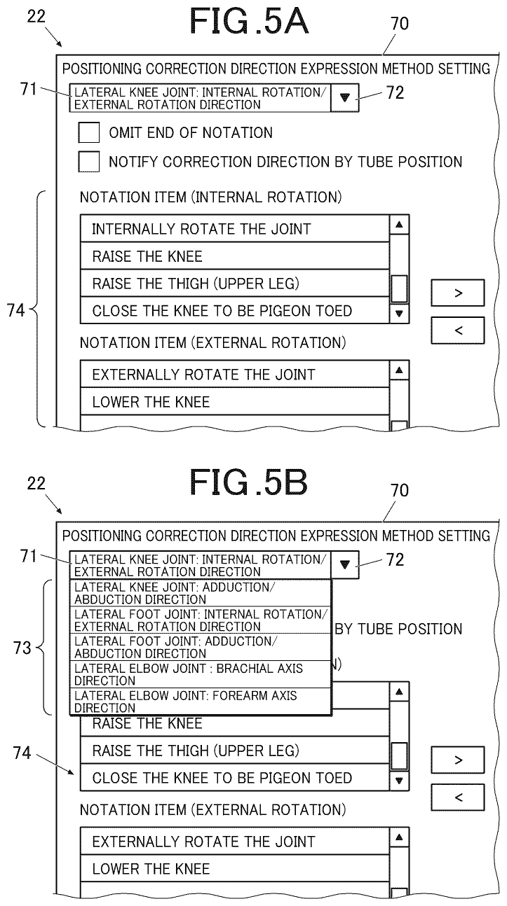

FIG. 5A shows an example of a configuration of the imaging support information setting screen 70. Note that hereinafter, the imaging support information setting screen 70 may be referred to as a setting screen 70.

The controller 20 causes the display part 22 to display the setting screen 70 with reference to the candidate table 23a in the storage section 23. The controller 20 sets the imaging support information I selected from among the plurality of replacement candidates on the setting screen 70 as the basis information to be the basis for the correction direction in correcting the positioning shift. The imaging support information may be set on the setting screen 70 at an arbitrary timing, for example, before the radiation image is captured.

The setting screen 70 is provided with a selection section 71 for selecting an imaging site and a correction direction in which the method of expressing the correction direction of positioning is desired to be changed. The case where, for example. “lateral knee joint: internal rotation/external rotation direction” is selected in the selection section 71 is illustrated in FIG. 5A. The selection section 71 is formed in, for example, a pull-down format. The selection section 71 is provided with, for example, a button 72 for developing pull-down.

FIG. 5B illustrates an example of a state in which the selection section 71 is developed in the setting screen 70. When the button 72 is pressed by the user, a pull-down list 73 is developed and displayed. In the pull-down list 73, a plurality of selection items such as “lateral: knee joint: adduction/abduction direction” are displayed in addition to “lateral knee joint: internal rotation/external rotation direction”. The user can select any item indicating the imaging site and the correction direction from the pull-down list 73.

In a display region below the selection section 71, a box 74 for selecting a specific replacement candidate from among a group of candidates for a plurality of pieces of imaging support information I is displayed. In the box 74, for example, when “lateral knee joint: internal rotation/external rotation direction” is selected by the selection section 71, a plurality of replacement candidates corresponding to the imaging support information I of the correction direction in internal rotation and external rotation are displayed. As candidates of the imaging support information I of the correction direction in the internal rotation. “internally rotate the knee”, “raise the knee”, “raise the thigh (upper leg)”, or “close the knee to be pigeon toed” is displayed. As replacement candidates for the method of expressing the correction direction in external rotation. “externally rotate the knee,” “lower the knee,” “lower the thigh (upper leg),” or “open the knee to be bowlegged” is displayed.

When the predetermined imaging support information I is selected by the user on the setting screen 70, the controller 20 stores the selected imaging support information I in the storage section 23 in association with the imaging site. In the present embodiment, the selected imaging support information I and the imaging site are stored in the imaging support information output table 23b in association with each other. Note that the first imaging support information Ia may be set as a default in advance in the initial setting of the imaging support information I, that is, at a stage before selection on the setting screen 70.

Note that the candidates for the new imaging support information I may be input by a user operation through an input section (inputter) provided on the setting screen 70. In other words, new first imaging support information Ia and second imaging support information Ib that are not displayed in the box 74 of the setting screen 70) can be registered in the candidate group of assist information. In the candidate table 23a of the storage section 23, the candidate for the imaging support information I input by the input section of the setting screen 70 is stored in association with the respective imaging sites. When new imaging support information I is selected on the setting screen 70), the controller 20 sets the selected imaging support information I as assist information for the correction direction of positioning.

### Example of Configuration of Imaging Support Information Output Table 23b

Next, an example of the configuration of the imaging support information output table 23b stored in the storage section 23 will be described. Hereinafter, the imaging support information output table 23b may be referred to as an output table 23b.

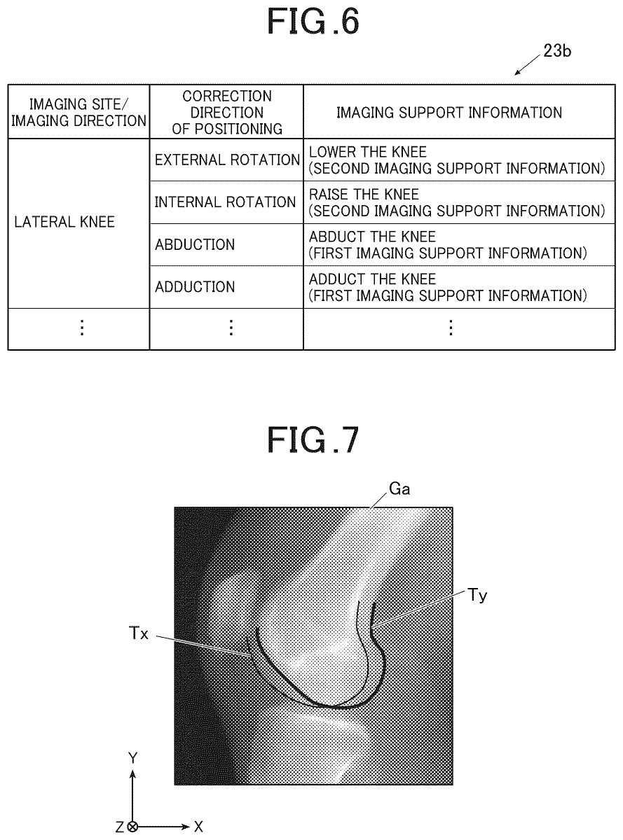

FIG. 6 illustrates an example of the configuration of the output table 23b. In the output table 23b, the imaging site, the correction direction of positioning, and the imaging support information I set as assist information of the correction direction of positioning are stored in association with each other. As the imaging support information I, for example, any assist information may be set by default. In this case, for example, the first imaging support information Ia including a word or a sentence of a technical term may be set as a default setting.

For example, when the imaging site and the imaging direction are the “knee joint side surface”, the four directions of external rotation, internal rotation, abduction, and adduction are exemplified as the correction direction of the positioning. In the present embodiment, the second imaging support information Ib “lower the knee” is set as the assist information for the correction direction of external rotation. As the assist information of the correction direction of internal rotation. “raise the knee” of the second imaging support information Ib is set. As the assist information of the correction direction of abduction, “abduct the knee” of the first imaging support information Ia is set. As the assist information of the correction direction of adduction. “Please adduction the knee” of the first imaging support information Ia is set.

Note that the output table 23b shown in FIG. 6 exemplifies a part of the imaging site. As with the “lateral knee”, the imaging support information I is set for the other imaging sites in association with the direction of positioning correction. Further, when the imaging support information I, which is the correction direction of the position of the predetermined imaging site, is changed on the setting screen 70, the imaging support information I of the corresponding portion of the output table 23b is also changed.

### About Machine Learning

FIG. 7 illustrates an example of an analysis result of a positioning shift in a radiation image Ga of a “lateral right knee joint” according to the related art. In FIG. 7, the horizontal direction (right-left direction) of the radiation image Ga is defined as an X direction, and the vertical direction (up-down direction) of the radiation image Ga is defined as a Y direction. In addition, an irradiation direction of the radiation R which is a direction orthogonal to the X direction and the Y direction of the radiation image Ga is set as a Z direction. In addition, it is assumed that the orthogonal coordinate system including the X direction, the Y direction, and the Z direction described above is applied to the radiation images, an imaging screen 80 on which the radiation images are displayed, and the like (described below).

In a case where there is a positioning shift in the radiation image Ga of the “lateral right knee joint”, a shift occurs at the medial condyle and the lateral condyle which are respectively positioned in the Z direction of the epiphysis, a structure inside the subject. In this case, since the radiation image Ga is two-dimensionally formed, a line indicating the medial condyle and a line indicating the lateral condyle of the “femoral condyle part” are displayed on the same plane. Therefore, as shown in FIG. 7, the medial condyle and the lateral condyle are displayed by two lines in the “femoral condyle part” of the radiation image Ga. These two lines are referred to as a first bone end Tx and a second bone end Ty. In the image analysis in the related art, it is not possible to specify which of the first bone end Tx and the second bone end Ty is a line indicating the medial condyle side positioned on the near side or a line indicating the lateral condyle side positioned on the far side. That is, the three-dimensional positional relationship between the two lines of the first and second bone ends Tx and Ty in the Z direction cannot be specified.

In the present embodiment, the machine learning model 23c is trained by machine learning using machine learning data by a learning device. The learning device is constituted by a computer, for example, and includes a processor such as a CPU and a GPU. GPU is an abbreviation for Graphics Processing Unit. The processor implements a predetermined machine learning function by executing a program stored in the memory such as the RAM, for example. The learning device may be a client device or a server device.

The machine learning model 23c outputs, as inference data, each of a line on the medial condyle side and a line on the lateral condyle side of the “femoral condyle part” in the radiation image Ga illustrated in FIG. 7. The machine learning model 23c may be, for example, a regression model, such as polynomial regression, multiple regression, support vector regression, or random forest regression. Further, the machine learning model 23c may be another model such as a neural network.

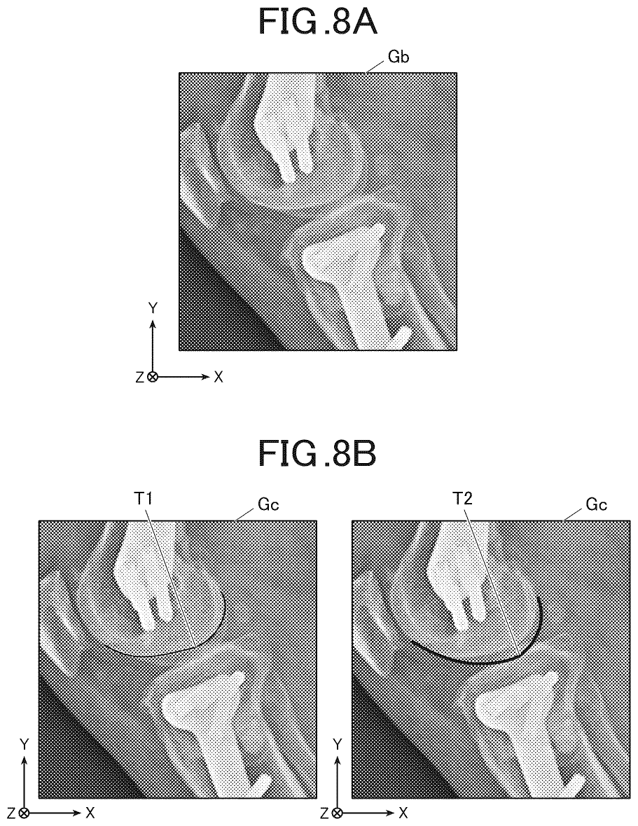

FIG. 8A illustrates an example of input data Gb used to train the machine learning model 23c. FIG. 8B illustrates an example of ground truth data Gc used for training the machine learning model 23c.

The machine learning data includes input data Gb to be input to the machine learning model 23c and ground truth data Gc to be output from the machine learning model 23c. For example, as shown in FIG. 8A, the input data Gb is radiation image data of the “lateral knee joint” in a case where the outer side of the right knee of the patient is imaged in contact with the imaging device 1. The input data Gb has a positioning shift in, for example, the “femoral condyle part”. As the input data Gb, a past radiation image of the patient obtained by actual imaging may be used.

As shown in FIG. 8B, the ground truth data Gc is the radiation image data of the “lateral knee-joint” in a case where the outer side of the right knee of the patient is imaged in contact with the imaging device 1. The ground truth data Gc includes information T1 on a medial condyle side line indicating a medial condyle region and information T2 on a lateral condyle side line indicating a lateral condyle region, which are ground truth for the input data Gb. Here, the medial condyle side line is a boundary between the medial condyle side of the femoral condyle part and the knee soft tissue portion. The lateral condyle line is the boundary between the lateral condyle side of the femoral condyle part and the knee soft tissue portion. Hereinafter, the information T1 on the medial condyle side line is referred to as medial condyle side line information T1, and the information T2 on the lateral condyle side line is referred to as lateral condyle side line information T2. The ground truth data Ge may be created by, for example, a user such as a radiologist. The user may specify the medial condyle side line information T1 and the lateral condyle side line information T2 from the radiation image, and tag the line information thereto. The labeled ground truth data Gc can be generated. Note that the medial condyle side line information T1 and the lateral condyle side line information T2 according to the present embodiment correspond to the three-dimensional structure information T.

The learning device performs machine learning using a data set including the input data Gb and the ground truth data Gc described above, and creates a trained machine learning model 23c. When the input data Gb of the “lateral right knee joint” is input, the machine learning model 23c outputs the medial condyle side line information T1 and the lateral condyle side line information T2 which are ground truth in a case where there is a positioning shift of the “femoral condyle part”. That is, the medial condyle and the lateral condyle of the “femoral condyle part” which is the epiphysis are distinguished into the medial condyle side line information T1 located on the near side and the lateral condyle side line information T2 located on the far side. The trained machine learning model 23c is stored in, for example, the storage section 23 of the imaging control device 2. The imaging control device 2 can identify the type of the positioning shift based on the medial condyle side line information T1 and the lateral condyle side line information T2 output from the machine learning model 23c. In this case, the imaging control device 2 can specify whether the “femoral condyle part” is internally rotated or externally rotated as the type of the shift in positioning.

The medial condyle of the femoral condyle part and the positional relationship in the Z direction of the medial condyle are learned using the medial condyle side line information T1 or the like actually designated by the user, but the learning method is not limited thereto. For example, as another learning method, respective coordinate points of the medial condyle and the lateral condyle of the femoral condyle part may be extracted, and the medial condyle and the positional relationship in the Z-axis direction of the medial condyle may be inferred by regression or the like of the extracted consecutive coordinate points.

Next, a case of specifying whether the “femoral condyle part” is adducted or abducted as the type of the positioning shift will be described. In this case, the machine learning model 23c is trained by machine learning using the ground truth data Gd different from the ground truth data Gc illustrated in FIG. 8B.

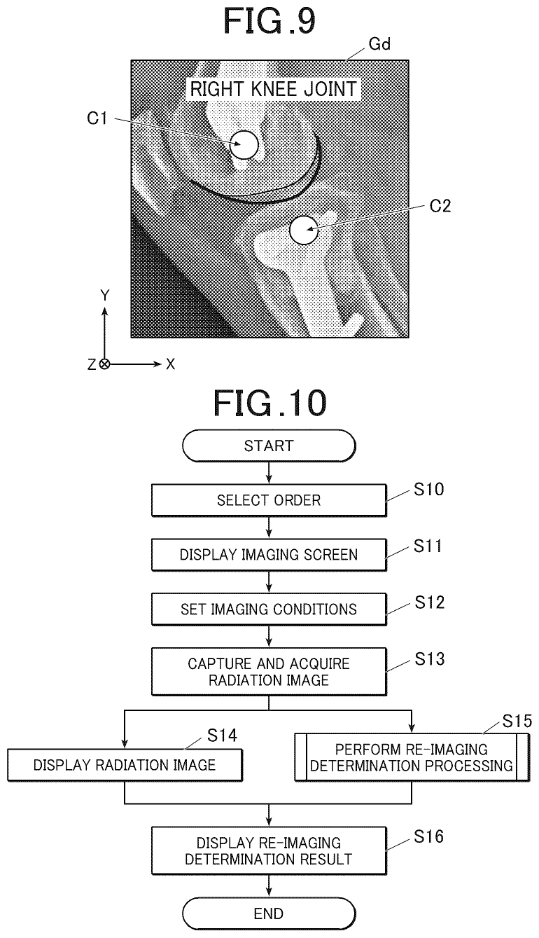

FIG. 9 illustrates an example of another ground truth data Gd used when the machine learning model 23c is trained. Note that the radiation image illustrated in FIG. 8A can be used as the input data Gb, and therefore. detailed description thereof is omitted.

The machine learning data includes input data Gb to be input to the machine learning model 23c and ground truth data Gd indicating ground truth for the output of the machine learning model 23c. As illustrated in FIG. 9), the ground truth data Gd includes information C1 indicating the center of femoral condyle part and information C2 indicating the center of crural condyle part which are ground truth for the input datum Gb. Hereinafter, the information C1 indicating the center of femoral condyle part is referred to as femoral condyle center information C1, and the information C2 indicating the center of crural condyle part is referred to as crural condyle center information C2. In addition, the ground truth data Gd includes joint information for specifying whether the radiation image of the input data Gb is a right knee joint or a left knee joint. The ground truth data Gd may be created by, for example, a user such as a radiologist. For example, the coordinates of the femoral condyle center information C1 and the coordinates of the crural condyle center information C2, which are the ground truth “femoral condyle part”, are determined by visual observation or the like by the user. Subsequently, two heat maps are created in accordance with the determined accuracies of the coordinates of the femoral condyle center information C1 and the coordinates of the crural condyle center information C2. The created heat map is used as the ground truth data Gd.

When the input data Gb of the “lateral knee joint” is input, the machine learning model 23c infers the femoral condyle center information C1, the crural condyle center information C2, and the joint information indicating which of the left and right knee joints is imaged as the ground truth. The trained machine learning model 23c is stored in, for example, the storage section 23 of the imaging control device 2. The imaging control device 2 identifies the type of positioning shift based on the femoral condyle center information C1 and the like output from the machine learning model 23c. In this case, the imaging control device 2 can specify whether the “femoral condyle part” is adducted or abducted as the type of the positioning shift.

Note that although the femoral condyle center information C1 and the like of the femoral condyle part are learned using the heat maps, a method other than this learning method may be used. As another learning method, learning may be performed using the center coordinates themselves of the femoral condyle part. Further, although the knee joint is specified by two alternatives of the right knee and the left knee, the knee joint may be specified by using other information such as a positional relationship between the femur and the patella. Alternatively, one machine learning model 23c may be used to infer all of the above-described medial condyle side line information T1, femoral condyle center information C1, and the like. Alternatively, a plurality of machine learning models 23c may be used. In that case, the medial condyle side line information T1 and the like may be inferred with one machine learning model 23c, and the femoral condyle center information C1 and the like may be inferred with another machine learning model 23c.

In the above-described example, the three-dimensional structures of the femoral condyle part on the “lateral right knee joint” in a case where the outer side of the right knee of the patient is imaged in contact with the imaging device 1 is learned, but the present invention is not limited thereto. For example, the three-dimensional structures of the femoral condyle part on the “lateral right knee joint” may be learned by using the radiation image of the “lateral right knee joint” in a case where the inner side of the right knee of the patient is imaged in contact with the imaging device 1. In that case, the medial condyle side line information T1 is positioned on the far side in the Z direction, and the lateral condyle side line information T2 is positioned on the near side in the Z direction. Further, the imaging site targeted for machine learning may be a region other than the “lateral right knee joint”. For example, the imaging site targeted for machine learning may be the lateral left knee joint, or may be another part such as the ankle joint or elbow joint.

### Operation Example of Imaging Control Device 2

Next, a flow in a case where the radiation image of the subject S is captured will be described. FIG. 10 is a flowchart illustrating an example of operations of the imaging control device 2 at the time of capturing the radiation image of the subject S. Hereinafter, a case in which the “lateral knee (right knee) joint” is imaged as the imaging site and the imaging direction of the radiation image will be described. In addition, it is assumed that the first imaging support information Ia or the second imaging support information Ib is set as the assist information on the correction direction of the positioning in the above-described setting screen 70 or the like by the user.

The communication section 24 of the imaging control device 2 receives the order information transmitted from the HIS/RIS 5 or the like. The user selects, for example, predetermined order information from the examination list displayed on the screen of the display part 22 of the imaging control device 2. The controller 20 acquires the order information selected by the user (step S10).

Upon acquiring the predetermined order information, the controller 20 allows the display part 22 to display the imaging screen 80 (step S11).

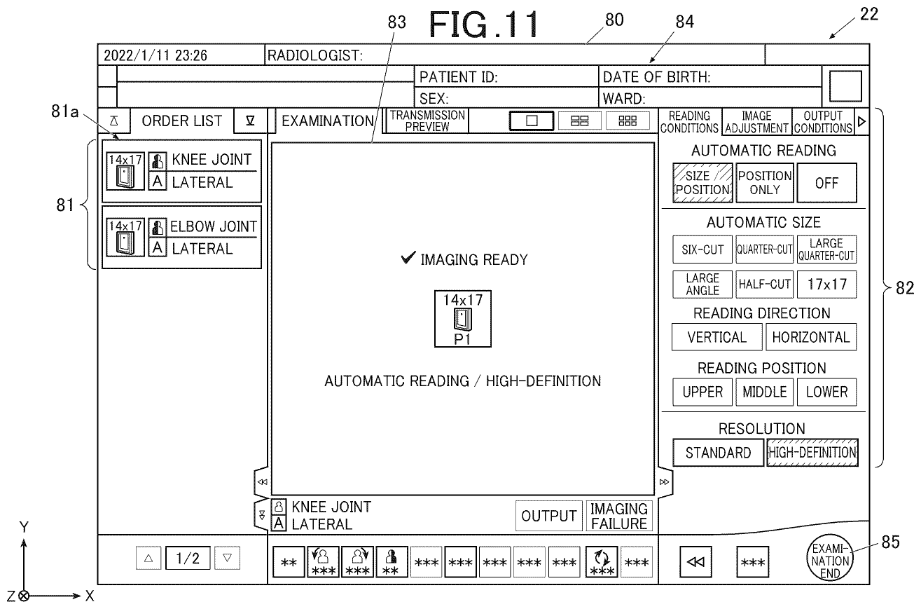

FIG. 11 shows an example of a configuration of an imaging screen 80 displayed on the display part 22 of the imaging control device 2. In FIG. 11, the horizontal direction (right-left direction) of the imaging screen 80 is defined as an X direction, and the vertical direction (up-down direction) of the imaging screen 80 is defined as a Y direction. In addition, an irradiation direction of the radiation R which is a direction orthogonal to the X direction and the Y direction of the imaging screen 80 is set as a Z direction. In addition, it is assumed that the orthogonal coordinate system including the X direction, the Y direction, and the Z direction described above is applied to the imaging screen 80, and the screen on which the radiation image G and the like (described below) are displayed, described below.

The imaging screen 80 is provided with an imaging selection area 81, a condition setting area 82, an image display area 83, a patient information display area 84, and an examination end button 85. In the imaging selection area 81, for example, the pieces of order information selected from the examination list are displayed. The order information includes, for example, imaging contents such as the imaging site and the imaging direction, and a thumbnail image indicating a captured radiation image. The condition setting area 82 is provided with, for example, a button for setting imaging conditions of a radiation image, a button for performing image adjustment of a captured radiation image, and the like. In the image display area 83, the radiation image captured by the imaging device 1 is displayed on the basis of the set imaging conditions or the like. Note that in FIG. 11, no radiation image is displayed in the image display area 83 because no radiation image has been captured.

When predetermined order information is selected in the imaging selection area 81 or the like, the controller 20 sets imaging conditions in each of the imaging device 1 and the generation device 3 (Step S12). The imaging conditions include image reading conditions to be set for the imaging device 1 and irradiation conditions to be set for the generation device 3. For example, the controller 20 sets the image reading conditions for the imaging device 1 on the basis of the imaging site, the imaging direction, and the like of the selected order information. Further, the controller 20 sets an irradiation condition in the generation device 3 based on the imaging site, the imaging direction, and the like of the selected order information.

The imaging conditions may be manually set by the user. Specifically, the controller 20 may set the image reading condition received by the input operation in the condition setting area 82 by the user in the imaging device 1. The controller 20 may set the radiation irradiation conditions received by user's input operation on the operation panel of the generation device 3 for the generation device 3.

Subsequently, when the switch 32 is turned on by the user, the controller 20 controls the imaging device 1, the generation device 3, and the like to capture a radiation image of the subject S (Step S13). The generation device 3 emits the radiation R to the imaging site of the subject S. The imaging device 1 detects the radiation R transmitted through the subject S from the generation device 3, and generates image data including the imaging site on the basis of the detected radiation R. The imaging device 1 transmits the generated image data to the imaging control device 2. The controller 20 acquires the radiation image based on the image data transmitted from the imaging device 1 (step S13).

Upon completion of Step S13, the process branches to Step S14 and Step S15. In the present embodiment, an example in which the processing of Step S14 and the processing of Step S15 are performed in parallel will be described, but the present invention is not limited thereto. For example, serial processing in which the processing of Step S14 and the processing of Step S15 are performed in order may be adopted. In this case, Step S15 may be performed first, and Step S14 and Step S16 which will be described later may be performed in combination.

First, the processing of Step S14 will be described. The controller 20 causes the acquired radiation image to be displayed in the image display area 83 of the imaging screen 80 (Step S14). In the present embodiment, the radiation image of the “lateral right knee joint” as the imaging site and the imaging direction is displayed in the image display area 83. In the order information 81a of the imaging selection area 81, a thumbnail image representing the captured radiation image is displayed. Upon completion of Step S14, the process proceeds to Step S16.

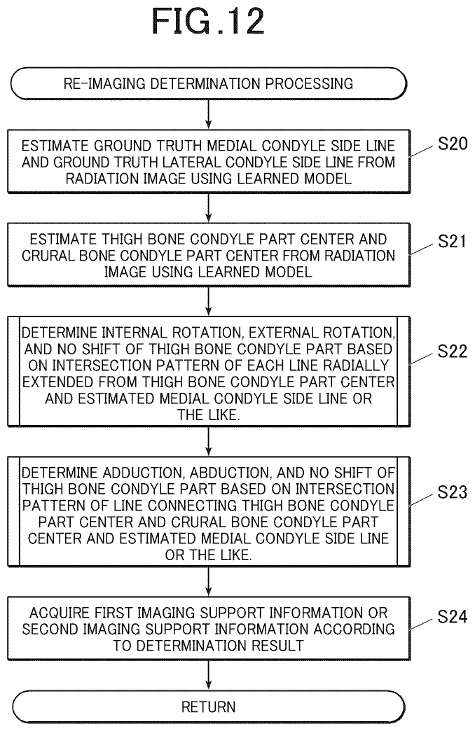

Subsequently, the processing in Step S15 which is branched from Step S13 will be described. The controller 20 executes re-imaging determination processing for obtaining information in order to determine whether or not re-imaging is necessary using the acquired radiation image. The controller 20 proceeds to the subroutine illustrated in FIG. 12.

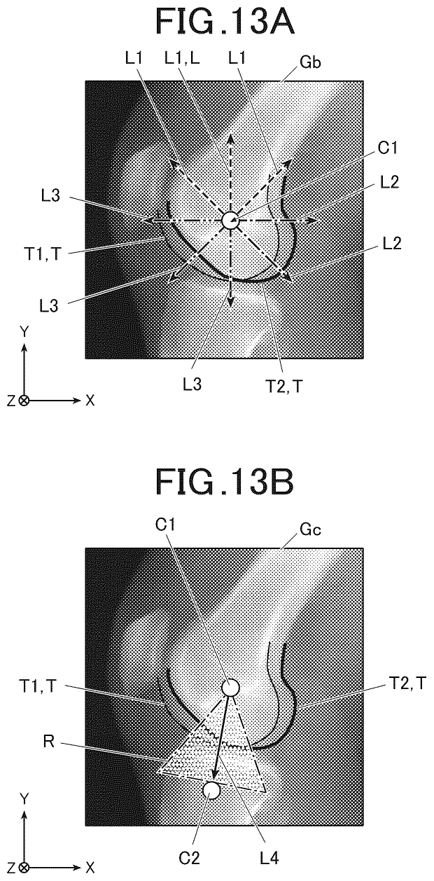

FIG. 12 is a flowchart illustrating an example of the operations of the imaging control device 2 during the re-imaging determination processing. FIG. 13A and FIG. 13B illustrate examples of the three-dimensional structure information T on the femoral condyle part in the radiation image G inferred by the machine learning model 23c.

Using the machine learning model 23c, the controller 20 infers the medial condyle side line information T1 and the lateral condyle side line information T2 on the femoral condyle part from the radiation image G obtained by imaging (Step S20). To be specific, the controller 20 inputs the radiation image G of the “lateral knee joint” to the machine learning model 23c. Based on the input radiation image G, the machine learning model 23c outputs ground truth medial condyle side line information T1 and ground truth lateral condyle side line information T2 of the femoral condyle part as inferred data. In FIG. 13A, etc., the medial condyle side line information T1 is indicated by a thin line, and the lateral condyle side line information T2 is indicated by a thick line. In this way, the controller 20 acquires the three-dimensional structure information T divided into the medial condyle side line information T1 positioned on the near side and the lateral condyle side line information T2 positioned on the far side.

Subsequently, the controller 20 uses the machine learning model 23c to infer the femoral condyle center information C1, the crural condyle center information C2, and the joint information from the radiation image G obtained by imaging (Step S21). To be specific, the controller 20 inputs the radiation image of the “lateral knee joint” to the machine learning model 23c. Based on the input radiation image G, the machine learning model 23c outputs femoral condyle center information C1 and crural condyle center information C2 of the femoral condyle part as inferred data. Furthermore, based on the input radiation image G, the machine learning model 23c outputs joint information indicating that the radiation image G captures the right knee joint. In this way, the controller 20 acquires the femoral condyle center information C1, the crural condyle center information C2, and the joint information. Note that the joint information may be acquired from examination order information transmitted from the HIS/RIS 5.

In the present embodiment. Step S20 and Step S21 have been described separately, but Step S20 and Step S21 may be one step. To be specific, the machine learning model 23c may output all of the medial condyle side line information T1, the lateral condyle side line information T2, the femoral condyle center information C1, the crural condyle center information C2, and the joint information on the basis of the input radiation image G. When Step S21 is completed, the process proceeds to Step S22.

The controller 20 determines whether the positioning shift is internal rotation or external rotation based on an intersection pattern between a line radially extending from the femoral condyle center information C1 and the medial condyle side line information T1 or the like (Step S22). In the present embodiment, processing of determining whether the shift of the positioning is internal rotation or external rotation is referred to as first determination processing.

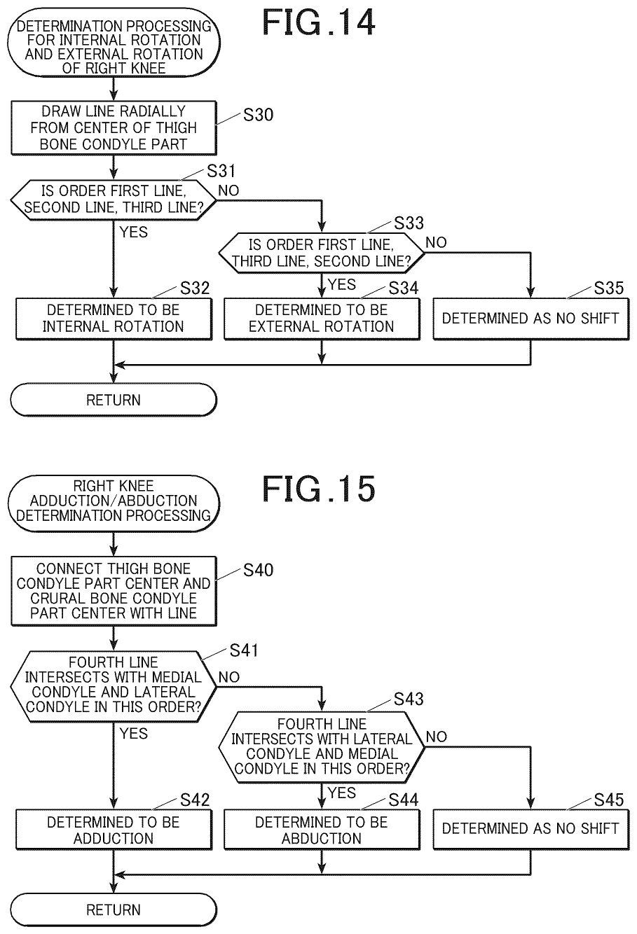

The controller 20 proceeds to the subroutine shown in FIG. 14 and functions as a first determination section to execute a first determination process. FIG. 14 is a flowchart illustrating an example of the first determination process.

The controller 20 extends a plurality of lines L radially from the inferred femoral condyle center information C1 (Step S30). To be specific, as shown in FIG. 13A, six lines L are radially extended at equal intervals in the circumferential direction from the femoral condyle center information C1. A line L that does not intersect with any of the medial condyle side line information T1 and the lateral condyle side line information T2 is referred to as a first line L1. In FIG. 13A, the first line L1 is indicated by a broken line. A line L that crosses the medial condyle side line information T1 and the lateral condyle side line information T2 in this order is referred to as a second line L2. In FIG. 13A, the second line L2 is indicated by a dash dot line. A line L that crosses the lateral condyle side line information T2 and the medial condyle side line information T1 in this order is referred to as a third line L3. In FIG. 13A, the third line L3 is indicated by a dash double-dot line. Note that the first line L1, the second line L2, and the third line L3 may be collectively referred to as lines L.

The controller 20 determines whether the order of the plurality of lines L is to be viewed clockwise or counterclockwise based on the inferred joint information. For example, the counterclockwise direction is associated with “right knee joint” of the joint information, and the clockwise direction is associated with “left knee joint” of the joint information. In the present embodiment, since the joint information is “right knee joint”, the order of the plurality of lines L is viewed counterclockwise. The controller 20 determines whether the first line L1, the second line L2, and the third line L3 are present in this order when viewed counterclockwise with reference to the first line L1 in the middle of FIG. 13A (Step S31). In a case where it is determined that the condition of Step S31 is satisfied, the controller 20 proceeds to Step S32.

When the order is the first line L1, the second line L2, and the third line L3, the controller 20 determines that the femoral condyle portion is internally rotated (Step S32). That is, the controller 20 determines that the femoral condyle part should be externally rotated in order to correct the positioning in the correct direction. Note that FIG. 13A is an example in which the femoral condyle part of the radiation image G is externally rotated, and Step S32 does not correspond to FIG. 13A.

On the other hand, in a case where it is determined that the condition of Step S31 is not satisfied, the controller 20 proceeds to Step S33. The controller 20 determines whether the first line L1, the third line L3, and the second line L2 are present in this order when viewed counterclockwise with reference to the first line L1 in the middle of FIG. 13A (Step S33). If the controller 20 determines that the condition of Step S33 is satisfied, the controller 20 proceeds to Step S34.

When the order is the first line L1, the third line L3, and the second line L2, the controller 20 determines that the femoral condyle part is externally rotated (Step S34). That is, the controller 20 determines that the femoral condyle part should be internally rotated in order to correct the positioning in the correct direction. Note that FIG. 13A is an example in which the femoral condyle part of the radiation image G is externally rotated, and Step S34 corresponds to FIG. 13A.

On the other hand, when determining that the condition of Step S33 is not satisfied, the controller 20 proceeds to Step S35. In this case, the controller 20 determines that the medial condyle side line information T1 and the lateral condyle side line information T2 of the femoral condyle part overlap with each other and the femoral condyle part is not shifted (Step S35). Note that the determination that the shift of the femoral condyle part does not occur is not limited to the case where the medial condyle side line information T1 and the lateral condyle side line information T2 completely overlap each other. When the overlap of the medial condyle side line information T1 and the lateral condyle side line information T2 is a shift amount within an allowable range, it may be determined that the shift of the femoral condyle part does not occur. When Step S35 ends, the controller 20 ends the subroutine of the first determination process, and proceeds to Step S23 in FIG. 12.

Note that although the case of the right knee joint has been described in the first determination processing, the type of positioning shift can be determined by similar processing also in the case of the left knee joint or the like. In the case of the left knee joint, the intersections between the plurality of lines L radially extending from the femoral condyle center information C1 and the medial condyle side line information T1 and the like are viewed clockwise with respect to the radiation image G. When the order is the first line L1, the second line L2, and the third line L3, the controller 20 determines that the femoral condyle part is internally rotated. When the order is the first line L1, the third line L3, and the second line L2, the controller 20 determines that the femoral condyle part is externally rotated.

Subsequently, the controller 20 determines whether the positioning shift is adduction or abduction based on the intersection pattern between the line connecting the femoral condyle center information C1 and the crural condyle center information C2 and the medial condyle side line information T1 or the like (Step S23). In the present embodiment, processing of determining whether the positioning shift is adduction or abduction is referred to as a second determination processing.

The controller 20 proceeds to the subroutine shown in FIG. 15 and functions as a second determination section to execute a second determination process. FIG. 15 is a flowchart illustrating an example of the second determination process.

As illustrated in FIG. 13B, the controller 20 connects the inferred femoral condyle center information C1 and the crural condyle center information C2 with a fourth line L4 (Step S40).

The controller 20 determines whether or not the fourth line L4 crosses the medial condyle side line information T1 and the lateral condyle side line information T2 in this order when the fourth line L4 is viewed from the femoral condyle center information C1 toward the crural condyle center information C2 (Step S41). In a case where it is determined that the condition of Step S41 is satisfied, the controller 20 proceeds to Step S42.

When the fourth line L4 intersects with the medial condyle side line information T1 and the lateral condyle side line information T2 in this order, the controller 20) determines that the femoral condyle part is adducted (Step S42). That is, the controller 20 determines that the femoral condyle part should be abducted in order to correct the positioning in the correct direction. Note that FIG. 13B is an example in which the femoral condyle part of the radiation image G is abducted, and Step S42 does not correspond to FIG. 13B.

On the other hand, when determining that the condition of Step S41 is not satisfied, the controller 20 proceeds to Step S43. The controller 20 determines whether or not the fourth line L4 intersects with the lateral condyle side line information T2 and the medial condyle side line information T1 in this order when viewing the fourth line L4 from the femoral condyle center information C1 toward the crural condyle center information C2 (Step S43). When the controller 20 determines that the condition of Step S43 is satisfied, the process proceeds to Step S44.

When the fourth line L4 intersects with the lateral condyle side line information T2 and the medial condyle side line information T1 in this order, the controller 20 determines that the femoral condyle part is abducted (Step S44). That is, the controller 20 determines that the femoral condyle part should be adducted in order to correct the positioning in the correct direction. Note that FIG. 13B is an example in which the femoral condyle part of the radiation image G is abducted, and Step S44 corresponds to FIG. 13B.

On the other hand, when the controller 20 determines that the condition of Step S43 is not satisfied, the process proceeds to Step S45. In this case, the controller 20 determines that the medial condyle side line information T1 and the lateral condyle side line information T2 of the femoral condyle part overlap with each other and no shift occurs (Step S45). The determination that no shift occurs also includes a case where the overlap between the medial condyle side line information T1 and the lateral condyle side line information T2 is within an allowable range regarding the shift amount. Note that step S45 is processing common to step S35 in FIG. 14, and therefore may be omitted. When Step S45 ends, the controller 20 ends the subroutine of the second determination process, and proceeds to Step S24 in FIG. 12.

Note that in the second determination processing, the case where the imaging site is the lateral right knee joint has been described, but also in the case where the imaging site is the lateral left knee joint or the like, the type of positioning shift can be determined by similar processing. A specific determination method is common to the case of the lateral right knee joint, and thus detailed description thereof will be omitted. Further, whether or not the femoral condyle part is adducted or the like is determined by using the fourth line L4 connecting the femoral condyle center information C1 and the crural condyle center information C2 shown in FIG. 13B, but the present invention is not limited thereto. The following determination method may be adopted. For example, a triangular virtual region R indicated by a broken line in FIG. 13B is assumed between the femoral condyle center information C1 and the crural condyle center information C2. Subsequently, a plurality of lines are drawn in the virtual region R in a direction from the femoral condyle center information C1 toward the crural condyle center information C2. At this time, in the plurality of lines, the number of lines intersecting in the order of the medial condyle side line information T1 and the lateral condyle side line information T2 is compared with the number of lines intersecting in the order of the lateral condyle side line information T2 and the medial condyle side line information T1. Finally, whether the femoral condyle portion is adducted or abducted may be determined based on which number is larger.

Subsequently, the controller 20 derives the first imaging support information Ia or the second imaging support information Ib associated with the correction direction of the position determined based on the three-dimensional structure information T in step S22 and step S23 (step S24). Specifically, when the second imaging support information Ib is set as the imaging support information corresponding to the determination result of the correction direction of the positioning, the controller 20 derives the second imaging support information Ib from the storage section 23. For example, in a case where the determination result of the first determination process is a determination that there is an external rotation, the controller 20 acquires the sentence “raise the knee” with reference to the output table 23b. In a case where the determination result of the second determination process is the abduction, the controller 20 acquires, for example, the sentence “lower the ankle” with reference to the output table 23b.

On the other hand, when the first imaging support information Ia is set as the imaging support information I corresponding to the determination result of the correction direction of the positioning, the controller 20 derives the first imaging support information Ia from the storage section 23. For example, in a case where the determination result of the first determination process is a determination that there is an external rotation, the controller 20 acquires the sentence “internally rotate the knee” with reference to the output table 23b. In a case where the determination result of the second determination process is the abduction, the controller 20 acquires, for example, the sentence “adduct the knee” with reference to the output table 23b.

Note that the controller 20 may acquire command information indicating that the positioning is normal when the determination result indicates that there is no positioning shift in Step S22 and Step S23. When the re-imaging determination process in Step S24 ends, the controller 20 ends the subroutine and returns to Step S16 shown in FIG. 10.

The controller 20 allows the image display area 83 of the display part 22 to display the acquired determination result of the re-imaging determination processing (step S16). Specifically, when deriving the second imaging support information Ib, the controller 20 allows the second imaging support information Ib and the three-dimensional structure information T indicating the shift region to be displayed on the imaging screen 80.

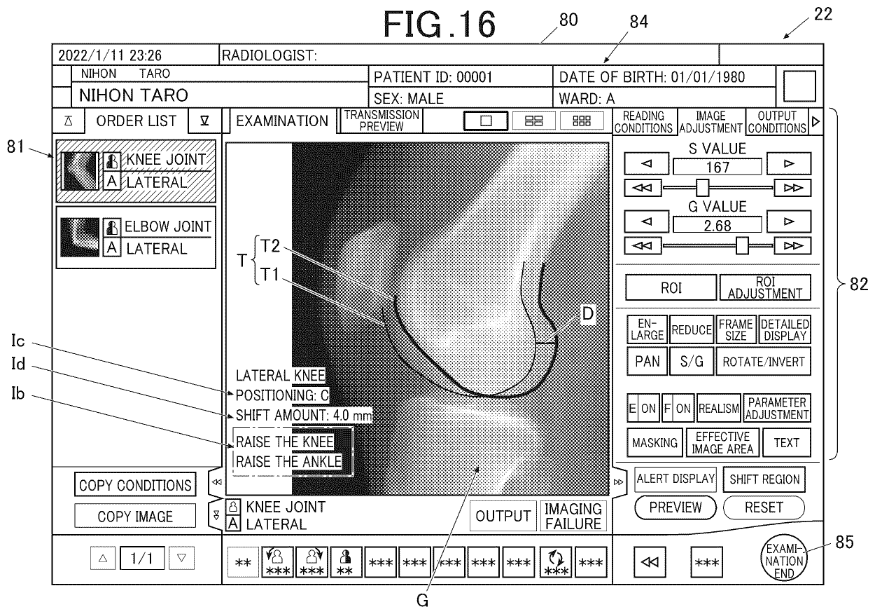

FIG. 16 shows an example of the second imaging support information Ib and the three-dimensional structure information T displayed on the imaging screen 80. In the image display area 83 of the imaging screen 80, the three-dimensional structure information T is displayed so as to be superimposed on the radiation image G. Here, in a case where there is a positioning shift in the radiation image G of the “lateral right knee joint”, a shift occurs between the medial condyle and the lateral condyle which are located on the Z direction side of the epiphysis of the femoral condyle part. In this case, since the radiation image G is two dimensionally formed, a line indicating the medial condyle and a line indicating the lateral condyle of the “femoral condyle part” are displayed on the same plane. Therefore, the medial condyle and the lateral condyle are displayed by two lines in the “femoral condyle part” of the radiation image G.

The three-dimensional structure information T includes, in the femoral condyle part of the radiation image G, the medial condyle side line information T1 indicating the medial condyle region and the lateral condyle side line information T2 indicating the lateral condyle region. The medial condyle side line information T1 and the lateral condyle side line information T2 which are ground truth in the femoral condyle part are displayed in the image display area 83 of the imaging screen 80, superimposed on the radiation image G. The medial condyle side line information T1 and the lateral condyle side line information T2 may be displayed in different colors. To be specific, the medial condyle side line information T1 may be displayed in red (thin line in FIG. 16), and the lateral condyle side line information T2 may be displayed in blue (thick line in FIG. 16). In addition, the medial condyle side line information T1 and the lateral condyle side line information T2 may be displayed with different line thicknesses, or may be displayed with a solid line and a broken line in a distinguished manner. In this case, it is preferable to set in advance a correspondence table or the like indicating a correspondence relationship between the “medial condyle side line information T1” and the “lateral condyle side line information T2” and the “color, thickness, line pattern (solid line, broken line)”. The correspondences may be a default setting, or may be appropriately set by a user after shipment. The user can quickly specify the three-dimensional positional relationship between the medial condyle side line information T1 and the lateral condyle side line information T2 by memorizing the correspondences in advance or confirming a correspondence table or the like indicating the correspondences. The correspondence table or the like indicating the correspondences may be displayed on the imaging screen 80.

In addition to the radiation image G obtained by imaging, the second imaging support information Ib based on the three-dimensional structure information T is displayed in the image display area 83 of the imaging screen 80). The second imaging support information Ib does not include technical terms, for example, and is constituted by words or sentences that are easy for the user to understand. Specifically, as shown in FIG. 16, when the direction of positioning correction is internal rotation, the sentence “raise the knee” is displayed as the second imaging support information Ib in the image display area 83. When the correction direction of the positioning is adduction, the sentence “raise the ankle” is displayed as the second imaging support information Ib in the image display area 83.

Above the imaging support information I, rank information Ic and shift amount information Id are displayed. The rank information Ic and the shift amount information Id are information for alerting the user that the positioning needs to be corrected and presenting detailed correction contents. In the present embodiment, the imaging support information I and the like are displayed in an empty space without the radiation image G in the image display area 83, but the present invention is not limited thereto.

The rank information Ic is information indicating a degree of the shift amount of the positioning shift by a rank. For example, when there is no positioning shift and re-imaging is not required, “positioning: A” is displayed as the rank information Ic. When the shift amount of the positioning shift is within an allowable range and re-imaging is not required, “positioning: B” is displayed as the rank information Ic. When the shift amount of the positioning shift exceeds the allowable range and re-imaging is required, “positioning: C” is displayed as the rank information Ic. In FIG. 16, the case of “positioning: C” as the rank information Ic is illustrated.

The shift amount information Id is information indicating a distance (shift width) in a predetermined direction between the inferred medial condyle side line information T1 and the inferred lateral condyle side line information T2 of the femoral condyle part. For example, when the shift amount D in the X direction in the three-dimensional structure information T between the medial condyle side line information T1 and the lateral condyle side line information T2 is “4 mm”, “shift amount: 4.0 mm” is displayed in the image display area 83 as the shift amount information Id. In FIG. 16, the shift amount D in the X direction is displayed as the shift amount information Id, but the present invention is not limited thereto. For example, the shift amount D in the Y direction may be displayed, or the shift amounts in both the X direction and the Y direction may be displayed.

In step S16, when the first imaging support information Ia is derived in the re-imaging determination processing illustrated in FIG. 12, the controller 20 allows the first imaging support information Ia and the three-dimensional structure information T to be displayed on the imaging screen 80.

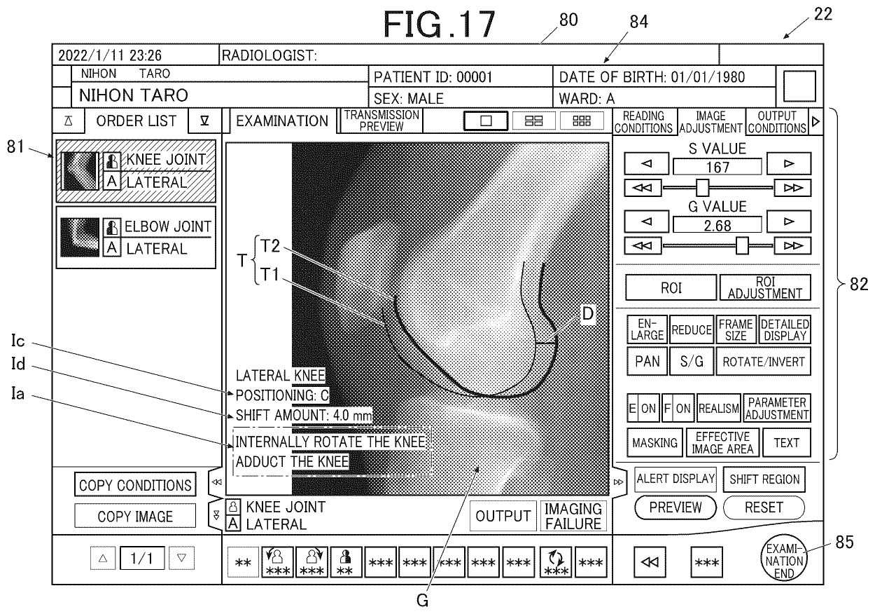

FIG. 17 shows an example of the first imaging support information Ia and the three-dimensional structure information T displayed on the imaging screen 80. In the image display area 83 of the imaging screen 80, the three-dimensional structure information T is displayed so as to be superimposed on the radiation image G. The three-dimensional structure information T includes, in the femoral condyle part of the radiation image G, the medial condyle side line information T1 indicating the medial condyle region and the lateral condyle side line information T2 indicating the lateral condyle region. The medial condyle side line information T1 and the lateral condyle side line information T2 which are ground truth in the femoral condyle part are displayed in the image display area 83 of the imaging screen 80, superimposed on the radiation image G. The medial condyle side line information T1 and the lateral condyle side line information T2 may be displayed in different colors. Note that the same display method as that in FIG. 16 can be applied to the details of the display method of the three-dimensional structure information T, and thus the detailed description will be omitted.

In addition to the radiation image G obtained by imaging, the first imaging support information Ia based on the three-dimensional structure information T is displayed in the image display area 83 of the imaging screen 80. Above the first imaging support information Ia, rank information Ic and shift amount information Id are displayed. The rank information Ic and the shift amount information Id have the same configuration and function as the rank information Ic and the shift amount information Id described in the second imaging support information Ib described above, and therefore, detailed description thereof is omitted.

The first imaging support information Ia includes, for example, words or sentences including technical terms. Specifically, when the direction of positioning correction is internal rotation, the sentence “internally rotate the knee” is displayed as the first imaging support information Ia in the image display area 83. In a case where the correction direction of the positioning is adduction, the sentence “adduct the knee” is displayed as the first imaging support information Ia in the image display area 83.

The user checks the first imaging support information Ia or the second imaging support information Ib and the three-dimensional structure information T displayed on the imaging screen 80. The first imaging support information Ia or the second imaging support information Ib displayed on the imaging screen 80 is a term with which the user is accustomed or an expression preferred by the user. Accordingly, the user can accurately guide the patient and correct the shift of the positioning based on the first imaging support information Ia or the second imaging support information Ib displayed on the imaging screen 80. When the positioning shift is resolved, the radiation image is re-captured.

On the other hand, in a case where it is determined that there is no positioning shift of the femoral condyle part, the controller 20 may display only the radiation image G in the image display area 83 of the imaging screen 80). In addition, in a case where it is determined that there is no positioning shift, the controller 20 may display “position: A” as the rank information Ic and “shift amount: 0 mm” as the shift amount information Id in the image display area 83. In addition, the controller 20 may display the sentence or the like indicating that there is no positioning shift in the image display area 83 as the imaging support information I.

In the present embodiment, the imaging support information I in two directions for supporting the correction of each shift amount is displayed on the imaging screen 80 as the assist information in the correction direction of the positioning, but the present invention is not limited thereto. For example, the imaging support information I corresponding to the shift amount in one direction may be displayed on the imaging screen 80. This is because a criterion for correcting positioning may vary depending on hospitals, facilities, or the like. Furthermore, depending on the patient, correction of positioning in one direction may be sufficient, or correction of positioning in two directions may be necessary. The selection of one direction or two directions in the correction direction of the positioning may be performed on the setting screen 70 or the like described above.

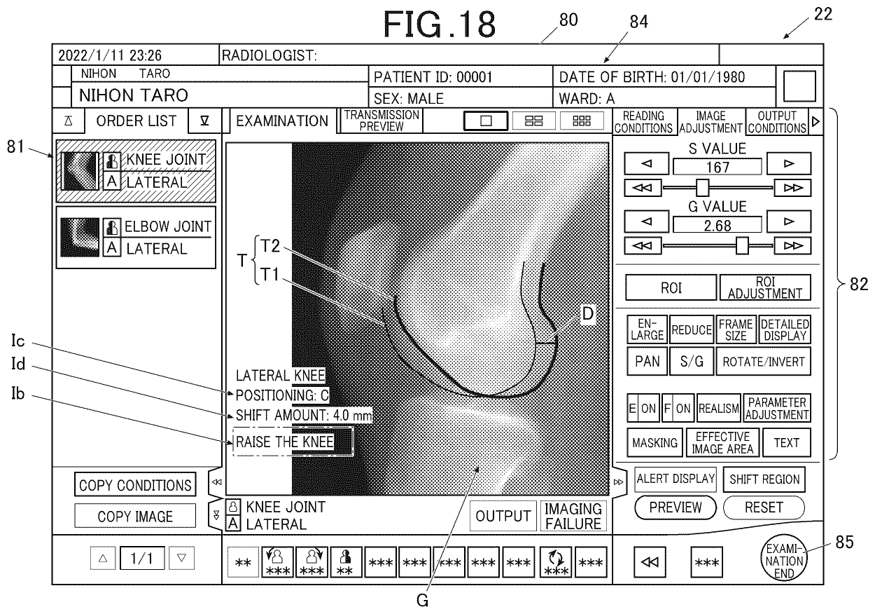

FIG. 18 shows an example of the second imaging support information Ib and the three-dimensional structure information T in the case of correcting the shift in positioning in one direction displayed on the imaging screen 80. In the image display area 83 of the imaging screen 80, three-dimensional structure information T indicating the shift in positioning is displayed so as to be superimposed on the radiation image G. As the three-dimensional structure information T, as shown in FIG. 18, medial condyle side line information T1 indicating a medial condyle region and lateral condyle side line information T2 indicating a lateral condyle region are displayed.

In addition to the radiation image G obtained by imaging, the second imaging support information Ib for correcting the shift in positioning is displayed in the image display area 83 of the imaging screen 80. Specifically, when the direction of positioning correction is internal rotation, the sentence “raise the knee” is displayed as the second imaging support information Ib in the image display area 83. Above the second imaging support information Ib, rank information Ic and shift amount information Id are displayed.

Furthermore, although the case where the posture of the patient who is the subject S is changed when the positioning is corrected has been described in the above-described embodiment, there is no limitation to this. For example, the positioning may be corrected by changing the position of the imaging device 1 using the imaging support information I for changing the position of the imaging device 1. As described above, the imaging support information I includes the first imaging support information Ia and the second imaging support information Ib. The first imaging support information Ia includes, for example, words or sentences including technical terms. Specifically, examples of the first imaging support information Ia include the sentence “move the radiation source (tube) toward the patella” and the like. The second imaging support information Ib is information obtained by changing the expression of the first imaging support information Ia, and is constituted by words or sentences that do not include technical terms and are easy for the user to understand. Specifically, examples of the second imaging support information Ib include the sentence “Move the radiation source (tube) to the front of the joint and above the joint”.

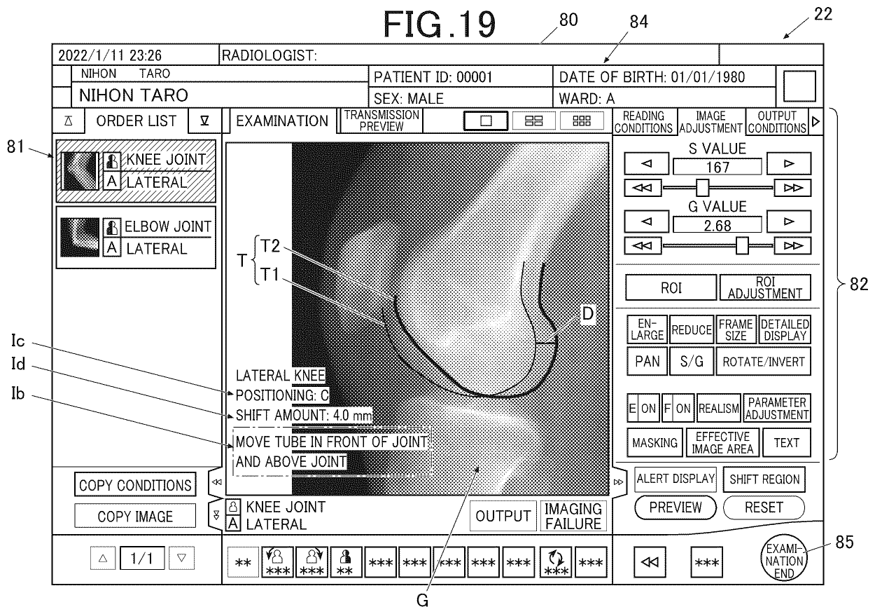

FIG. 19 is a diagram illustrating an example of the second imaging support information Ib and the three-dimensional structure information T for changing the position of the imaging device 1 displayed on the imaging screen 80) according to the present embodiment. In addition to the radiation image G obtained by imaging, the second imaging support information Ib for changing the position of the imaging device 1 is displayed in the image display area 83 of the imaging screen 80. Specifically, when the positioning needs to be corrected, the sentence “Move the radiation source (tube) to the front of the joint and above the joint” is displayed as the second imaging support information Ib in the image display area 83. Above the second imaging support information Ib, rank information Ic and shift amount information Id are displayed. Thus, the user can change the imaging position with respect to the subject S by adjusting the position of the imaging device 1, thereby indirectly correcting the positioning. In addition, in a case where the first imaging support information Ia is selected as the imaging support information I, although not illustrated, the sentence “move the radiation source (tube) toward the patella” is displayed in the image display area 83.

As described above, according to the present embodiment, for example, it is possible to select the second imaging support information Ib that the user is accustomed to using or that matches the expression of the user's preference from among a plurality of candidate groups on the setting screen 70. In addition, some users are more accustomed to using the first imaging support information Ia including technical terms. In this case, the first imaging support information Ia can be selected on the setting screen 70. That is, in the present embodiment, assist information on a direction in which positioning is corrected can be freely selected for each user. Thus, the user can correct the positioning with the assistance of the word or the sentence that can be more easily understood, so that the patient can be accurately guided. As a result, the speed of radiation imaging can be increased, and the burden on the patient during positioning can be reduced.

### Other Embodiment 1

In Other Embodiment 1, positioning support is performed using an optical camera image captured by an optical camera. Note that hereinafter, differences from the above-described embodiment will be mainly described, and description of points common to the above-described embodiment will be omitted. Furthermore, in the description of Other Embodiment 1, the same parts as those in the above-described embodiment will be described with the same reference symbols.

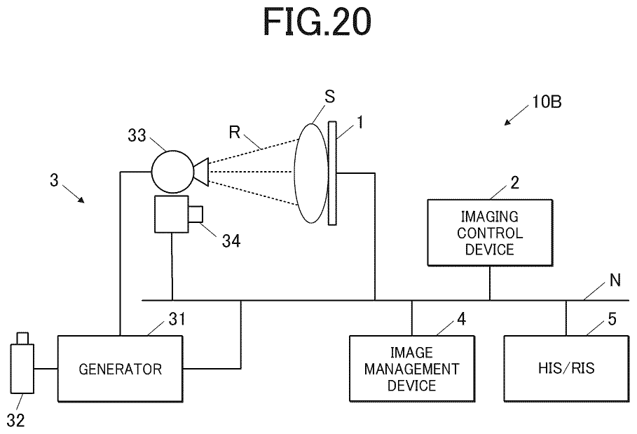

FIG. 20 is a diagram illustrating a schematic configuration of an imaging support system 10B according to Other Embodiment 1, The imaging support system 10B includes a radiographic imaging device 1, an imaging control device 2, a radiation generation device 3, an image management device 4, and a HIS/RIS 5. The generation device 3 includes a generator 31, a switch 32, a radiation source 33, and an optical camera 34. The optical camera 34 and the like are connected to the imaging control device 2 via a network N.

For example, the optical camera 34 is arranged side by side at a position adjacent to the radiation source 33. The radiation source 33 and the optical camera 34 may be integrally mounted in one housing. The optical camera 34 optically captures an image of the subject S at a timing before capturing a radiation image of the subject S. The optical camera 34 transmits the captured optical camera image corresponding to the positioning of the patient to the imaging control device 2. The optical camera image includes a still image or continuously captured dynamic images.

The imaging control device 2 determines the presence or absence, the type, and the like of the positioning shift based on the optical camera image acquired from the optical camera 34. For example, the imaging control device 2 inputs the acquired optical camera image to the machine learning model 23c to infer the three-dimensional structure information T such as the medial condyle side line information T1 indicating the shift region of the structure of the subject S. The imaging control device 2 specifies the type of shift in positioning based on the three-dimensional structure information T or the like, and acquires the imaging support information I corresponding to the type of shift. The imaging support information I is the first imaging support information Ia or the second imaging support information Ib as described above. The imaging control device 2 allows, for example, the display part 22 to display the acquired imaging support information I, three-dimensional structure information T, and the like. A user such as a radiologist can recognize the three-dimensional positional relationship of the shift region from the three-dimensional structure information T, and can easily understand the direction of positioning correction from the imaging support information I. In a case in which the shift in positioning is resolved, the user proceeds to the next step, that is, capturing the next radiation image of the subject S.

According to Other Embodiment 1, similarly to the above-described embodiment, the user such as a radiologist can specify the three-dimensional positional relationship in the “femoral condyle part” by confirming the three-dimensional structural information T displayed on the imaging screen 80. Thus, the user can accurately grasp the direction in which the positioning is to be corrected. As a result, the speed of radiation imaging can be increased, and the burden on the patient during positioning can be reduced. Furthermore, according to another embodiment 1, the positioning of the patient is imaged using the optical camera 34 before the radiation imaging. Therefore, when there is a positioning shift in the optical camera image, the positioning can be corrected before the radiation imaging. Thus, the number of times of re-imaging can be reduced, and the burden on the patient can also be reduced by reducing the total amount of exposure of the patient.

Although the preferred embodiments of the present disclosure have been described in detail with reference to the accompanying drawings, the technical scope of the present disclosure is not limited to such examples. Furthermore, those to which various modification examples and improvements have been applied naturally belong to the technical scope of the present disclosure within the category of the technical idea described in the scope of the claims of those skilled in the art.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2023-169682, filed on Sep. 29, 2023, including description, claims, drawings and abstract is incorporated herein by reference.

What is claimed is:

1. An imaging support device comprising:
a hardware processor,
wherein the hardware processor,
acquires first imaging support information which is first information for changing a position of a subject or an imaging device configured to image a medical image of the subject,
changes the first imaging support information to second imaging support information which is second information for changing a position of the subject or the imaging device, and
outputs the second imaging support information.

2. The imaging support device according to claim 1, wherein the medical image is a radiation image.

3. The imaging support device according to claim 1, wherein the medical image is an optical camera image.

4. The imaging support device according to claim 1, wherein the hardware processor changes an expression of the first imaging support information to obtain the second imaging support information.

5. The imaging support device according to claim 1,
further comprising a storage that stores a plurality of replacement candidates corresponding to a word or a sentence before replacement included in the first imaging support information,
wherein the hardware processor,
sets, from among the plurality of replacement candidates, a replaced word or sentence corresponding to a word or sentence before replacement included in the first imaging support information,
changes the first imaging support information to the second imaging support information based on the set replaced word or sentence, and
outputs the second imaging support information.

6. The imaging support device according to claim 5,
further comprising an inputter to which a user inputs a replacement candidate corresponding to the word or the sentence included in the first imaging support information,
wherein the storage stores the replacement candidate input to the inputter.

7. The imaging support device according to claim 5,
further comprising an inputter to which a user inputs a replacement candidate corresponding to the word or the sentence included in the first imaging support information,
wherein the hardware processor changes the first imaging support information to the second imaging support information based on the replacement candidate input to the inputter.

8. The imaging support device according to claim 5, wherein the word or the sentence included in the first imaging support information is at least one of adduction, abduction, internal rotation, and external rotation.

9. The imaging support device according to claim 5,
further comprising a storage that stores first plurality of replacement candidates and second plurality of replacement candidates corresponding to the word or the sentence before replacement included in the first imaging support information,
wherein the hardware processor,
sets, from the first plurality of replacement candidates, a replaced word or sentence after a first replacement corresponding to the word or the sentence before the first replacement included in the first imaging support information,
sets, from the second plurality of replacement candidates, a replaced word or sentence after a second replacement corresponding to the word or the sentence before the second replacement included in the first imaging support information, and
changes the first imaging support information to the second imaging support information based on the set word or sentence after the first replacement, and changes the first imaging support information to the second imaging support information based on the set word or sentence after the second replacement.

10. The imaging support device according to claim 2, wherein
the first imaging support information is first re-imaging support information that is information to support re-imaging of the radiation image, and
the second imaging support information is second re-imaging support information that is information to support re-imaging of the radiation image.

11. The imaging support device according to claim 10, wherein the hardware processor,
determines a shift of a predetermined region of the radiation image, and
derives the first re-imaging support information or the second re-imaging support information based on a determination of the shift.

12. The imaging support device according to claim 11, wherein as the second re-imaging support information, information for changing a position of a site of a subject related to the shift or a position of an imaging device related to the shift is output.

13. The imaging support device according to claim 11, wherein the hardware processor,
determines a first shift of a predetermined region in the radiation image,
determines a second shift of a predetermined region in the radiation image, and
derives the first re-imaging support information or the second re-imaging support information based on a determination of the first shift and a determination of the second shift.

14. The imaging support device according to claim 13, further comprising a storage that stores a first replacement candidate related to the first shift and a second replacement candidate related to the second shift.

15. A non-transitory computer-readable recording medium storing a program that causes a computer to perform:
acquiring first imaging support information which is first information for changing a position of a subject or an imaging device configured to image a medical image of the subject,
changing the first imaging support information to second imaging support information which is second information for changing a position of the subject or the imaging device, and
outputting the second imaging support information.

16. The recording medium according to claim 15, wherein an expression of the first imaging support information is changed to obtain the second imaging support information.

17. The recording medium according to claim 15, wherein,
from among the plurality of replacement candidates corresponding to a word or sentence before replacement included in the first imaging support information, a replaced word or sentence corresponding to a word or sentence before replacement included in the first imaging support information is set,
the first imaging support information is changed to the second imaging support information based on the set replaced word or sentence, and
the second imaging support information is output.

18. An imaging support method comprising:
acquiring first imaging support information which is first information for changing a position of a subject or an imaging device configured to image a medical image of the subject,
changing the first imaging support information to second imaging support information which is second information for changing a position of the subject or the imaging device, and
outputting the second imaging support information.

19. An imaging support system comprising:
an imaging device that images a subject or a medical image of the subject, and
the imaging support device according to claim 1.
